(19) United States
(12) Patent Application Publication
(10) Pub. No.: US 2012/0158136 A1
(43) Pub. Date: Jun. 21, 2012

(54) Surgical Implant

(75) Inventors: Cyril Voisard, Niederbipp (CH); Adrian Baumgartner, Oberdorf (CH); Christian Brunner, Basel (CH)

(73) Assignee: SYNTHES USA, LLC, West Chester, PA (US)

(21) Appl. No.: 13/315,405

(22) Filed: Dec. 9, 2011

Related U.S. Application Data
(60) Provisional application No. 61/423,916, filed on Dec. 16, 2010.

(51) Int. Cl. A61F 2/02

(52) U.S. Cl. 623/11.11

(57) ABSTRACT

A surgical implant comprises a body having a compressed state and an uncompressed state. An envelope contains the body in at least the compressed state. The envelope forms an air-tight seal around the body in the compressed state and is water-soluble or degradable in body fluids.

### CROSS-REFERENCE TO RELATED APPLICATIONS

This application claims the benefit of U.S. Provisional Patent Application No. 61/423,916 filed Dec. 16, 2010 entitled “Surgical Implant”, incorporated by reference herein in its entirety.

### BACKGROUND OF THE INVENTION

The present invention generally relates to surgical implants. More particularly, the present invention relates to expandable surgical implants.

### BRIEF SUMMARY OF THE INVENTION

In certain surgical procedures, it is desirable to have an implant that expands after insertion into the body. For example, in minimally invasive surgery, an expandable implant can be used to reduce the size of the entry incision. Expandable implants may also be used to conform to a patient's anatomy or as an anchoring device.

In one embodiment there is a surgical implant comprising a body having a compressed state and an uncompressed state; and an envelope containing the body in at least the compressed state, the envelope forming an air-tight seal around the body in the compressed state and the envelope being water-soluble and/or degradable in body fluids. In one embodiment, the body includes a plurality of pores and/or cavities. In one embodiment, the pores or cavities of the body in an uncompressed state have a size of 10 μm to 2 mm. In one embodiment, a vacuum in the pores and/or cavities in the compressed state is 10 mbar or less. In one embodiment, the envelope fully contains the body in the uncompressed state.

In one embodiment, the envelope includes a one-way valve for evacuation of air from the body from the uncompressed state to the compressed state. In one embodiment, only part of a total area of the envelope is water-soluble or degradable in body fluids. In one embodiment, a remaining part of the total area of the envelope comprises a high strength polymer. In a further embodiment, the implant includes a protective sheath at least partially surrounding the envelope and comprised of a thermoplastic material. In one embodiment, the thermoplastic material is polylactide (PLA) or polycaprolactone (PCL).

In one embodiment, the body is comprised of a polymeric material. In one embodiment, the body is comprised of a foam material. In one embodiment, the envelope is comprised of polyvinyl alcohol (PVA) or methylcellulose. In one embodiment, the envelope includes one or more regions comprised of a material having a dissolution rate D, a remainder of the envelope being comprised of material having a dissolution rate d<D. In one embodiment, the envelope is surrounded by a protective sheath made of a material not permeable to water. In one embodiment, the envelope has a minimum thickness of 10 μm. In one embodiment, the envelope has a maximum thickness of 500 μm. In one embodiment, the body in the uncompressed state has a degree of porosity larger than 80%. In one embodiment, the body has a porosity and a degree of compression of 5±2% when the porosity is 80% and a degree of compression of 20±5% when the porosity is 95%.

### BRIEF DESCRIPTION OF THE DRAWINGS

Several embodiments of the invention will be described in the following by way of example and with reference to the accompanying drawings. It should be understood, however, that the invention is not limited to the precise arrangements and instrumentalities shown:

FIG. 1 is a schematic cross sectional view of an implant in accordance with an exemplary embodiment of the present invention;

FIG. 2 is a schematic cross sectional view of an implant in accordance with another exemplary embodiment of the present invention;

FIG. 3 is a perspective view of an uncompressed body of the implant shown in FIG. 1;

FIG. 4 is a schematic cross sectional view of an implant in accordance with an exemplary embodiment of the present invention;

FIG. 5 is a schematic lateral view of an implant in accordance with an exemplary embodiment of the present invention being implanted between two vertebrae;

FIG. 6 is a schematic lateral view of the implant according to FIG. 5 in the implanted state;

FIG. 7 is a schematic lateral view of an implant in accordance with an exemplary embodiment of the present invention attached to a bone anchor;

FIG. 8 is a schematic lateral view of the implant according to FIG. 7 in the implanted state;

FIG. 9 is a schematic cross sectional view of an implant in accordance with an exemplary embodiment of the present invention for internal locking of an intramedullary nail;

FIG. 10 is a schematic cross sectional view of the implant shown in FIG. 9 in the implanted state;

FIG. 11 is an enlarged perspective view of a portion of the body of an implant in accordance with an exemplary embodiment of the present invention shown in the expanded configuration; and

FIG. 12 is a perspective view of an implant and envelope in accordance with an exemplary embodiment of the present invention shown in the compressed configuration.

### DETAILED DESCRIPTION OF THE INVENTION

The materials used for expandable implantable devices are typically made either from metals or from dense polymeric materials which, due to their nature, do not allow for compression and expansion of the material itself. A stent is an example. The constraining means of such devices may also be purely mechanical, like springs or memory metals, and may severely limit the possible degree of constraint and consequently of the subsequent expansion.

In one embodiment, there is a surgical implant that can expand after implantation.

In one embodiment, there is a surgical implant that includes a body having a compressed state and an uncompressed state. An envelope may contain the body in at least the compressed state. The envelope may form an air-tight seal around the body in the compressed state and is water-soluble and/or degradable in body fluids.

In one embodiment, there is a surgical implant that includes: A) a compressed polymeric body having open pores or cavities which have been evacuated by the action of an external vacuum applied to the uncompressed polymeric body; and B) an envelope containing the compressed polymeric body in an air-tight and compressed manner, the envelope being water-soluble or degradable in body fluids. In one embodiment, at least a portion of the pores are interconnected and open to the outside of the body in order that they can be evacuated by application of an external vacuum leading to a shrinking of the body. Further, in one embodiment, the open porosity of the polymeric body allows an instantaneous free exchange with the surrounding environment.

The advantages obtainable with embodiments of the implant may include the following:
achieving a larger pre-stress by compressing the implant using a vacuum compared to a purely mechanical compression;
achieving a larger strain using a vacuum; and
compressing the implant to a significantly much smaller shape.

In one embodiment, after solving or degrading the envelope in vivo, air is allowed to penetrate in the compressed polymer and the polymer re-expands. With appropriate chemical design the reaction kinetics can be tuned.

In some embodiments, the implant is be used to support the fixation of osteosynthesis devices like plates or nails or to fill bone voids. Due to the high compression ratio of the body of the implant, the implant may be minimally invasive inserted, e.g. through an adequate tube.

The body of the implant according to one embodiment of the invention can comprise a highly porous piece of elastomer, silicone or biodegradable material like materials from the poly-lactide or poly-caprolactide family. The envelope may comprise a thin film of polyvinyl alcohol (PVA), starch or methylcellulose. In one embodiment, the envelope completely surrounds and seals in the body of the implant. In one embodiment, the envelope covers only a portion of the body of the implant. In one embodiment, the envelope sounds a portion of the body of the implant leaving at least one surface exposed (e.g., a cylindrical body may be covered by an envelope around the curved sidewall but left open on the top and/or bottom). In one embodiment, the envelope covers at least enough of the body of the implant to retain the body in a compressed configuration. In one embodiment, the envelope is opaque. In other embodiments, the envelope is at least partially transparent. The surgical implant may have any shape including, for example, a highly compressed cylinder that expands after insertion and package dilution.

In one embodiment, the envelope is bioresorbable. In one embodiment, the envelope comprises polyvinyl alcohol (PVA) or methylcellulose. In such a configuration, the release of lactic acid may be avoided.

In a further embodiment, only part of the total area of the envelope is water-soluble or degradable in body fluids. By this means the advantage may be achieved that the opening process of the envelope is much faster. For example, the resorbable part may be limited to a small cork only or to stripes along the envelope. In one embodiment, a part of the envelope can resorb slower than the cork or stripes and may comprise biocompatible elastomers with plastic deformation ability. Examples for such materials are poly-carbonate urethane or silicone.

In a further embodiment of the implant, the remaining part of the total area of the envelope comprises a high strength polymer which is easier to process and to handle.

In again a further embodiment of the implant, the envelope has one or more regions made of a material having a dissolution rate D, the remaining part of the envelope being made of material having a dissolution rate d<D. In such a configuration:
the regions with the higher dissolution rate may dissolve more quickly than the regions with the lower dissolution rate; and
the higher mechanical strength may allow the use of a variety of suitable materials.

In another embodiment of the implant, the envelope has a valve for its evacuation.

In again another embodiment of the implant, the envelope is surrounded by a protective sheath made of a material not permeable to water. The protective sheath protects the dissolvable envelope from prematurely dissolving before implantation. In again another embodiment of the implant, said protective sheath comprises a thermoplastic material. In yet another embodiment of the implant, said thermoplastic material is PLA or PCL, preferably in a dense form.

In a further embodiment of the implant, the envelope has a minimum thickness of about 10 μm, preferably of about 100 μm. In a further embodiment of the implant, the envelope has a maximum thickness of about 500 μm, preferably of about 300 μm.

In again a further embodiment of the implant, the compressed body has in its uncompressed state a degree of porosity larger than about 80%. In still a further embodiment of the implant, the pores or cavities of the uncompressed body have a size of about 10 μm to about 2 mm. In some embodiments, the pores are larger than about 1 mm. In another embodiment of the implant, the vacuum in said pores or cavities is about 10 mbar or less. In again another embodiment of the implant, the degree of compression of the compressed body is about 5±2% for 80% porosity and is about 20±5% for 95% porosity.

Several methods may be used for manufacturing the implant, e.g. by impregnation of a porous body with CO₂ under high pressure followed by rapid decompression, bubbling with air in the melt, impregnation of water of the sealed polymer, consolidation of polymer granules mixed with coarse filler material like SiO₂, TiO₂, HA.

The envelope may be prepared as follows: dipping the porous body in a highly viscous melt of the dissolvable material, or using a self-standing bag made of the dissolvable material. The bag may be coated with a second material, dissolving much slower to protect it from a too fast dissolution (protective sheath). The air is evacuated from the porous body and from the bag. The thermoplastic material of the bag may be sealed by welding at a neck portion thereof.

In at least some embodiments, the implant may be used in the following applications:

A) as a bone anchor:

A material is chosen for the envelope which can dissolve by the action of water within seconds. After having been implanted into the bone cavity the envelope of the bone anchor dissolves quickly and by the expansion of the compressed body the bone anchor is firmly held in the bone cavity.

B) For minimally invasive surgical procedures:

The various implants can be introduced laparoscopically in their small, compressed shape.

The envelope will dissolve after some time—according to the envelope material chosen—and the implant will expand.

According to a further embodiment of the invention, there is provided a method for replacing at least a portion of a nucleus pulposus with an implant according to embodiments of the invention in the form of an intervertebral implant.

According to a further embodiment of the invention, there is provided a method for attaching a suture to bone and soft tissue with an implant according to embodiments of the invention in the form of an anchor.

According to yet a further embodiment of the invention, there is provided a method for vertebroplasty with an implant according to embodiments of the invention.

According to another embodiment of the invention, there is provided a method for treating osteoporosis with an implant according to embodiments of the invention.

According to another embodiment of the invention, there is provided a method for bone fixation with an implant according to embodiments of the invention.

According to yet another embodiment of the invention, there is provided a method for treating spine deformations with an implant according to embodiments of the invention in the form of an interspinous spacer.

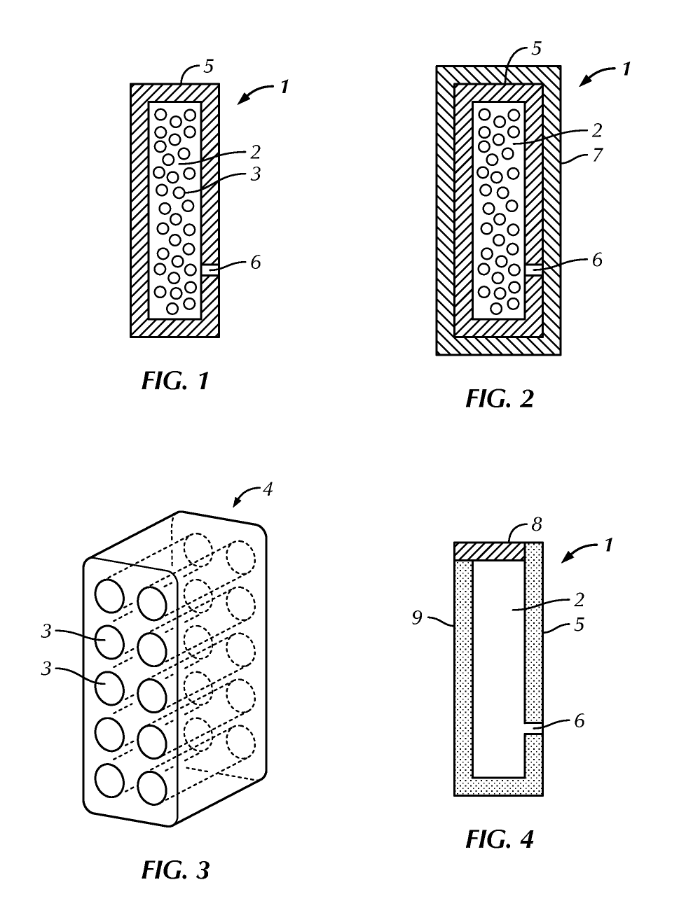

Referring to the drawings in detail, wherein like reference numerals indicate like elements throughout, there is shown in FIGS. 1-12 implants, generally designated 1, in accordance with exemplary embodiments of the present invention.

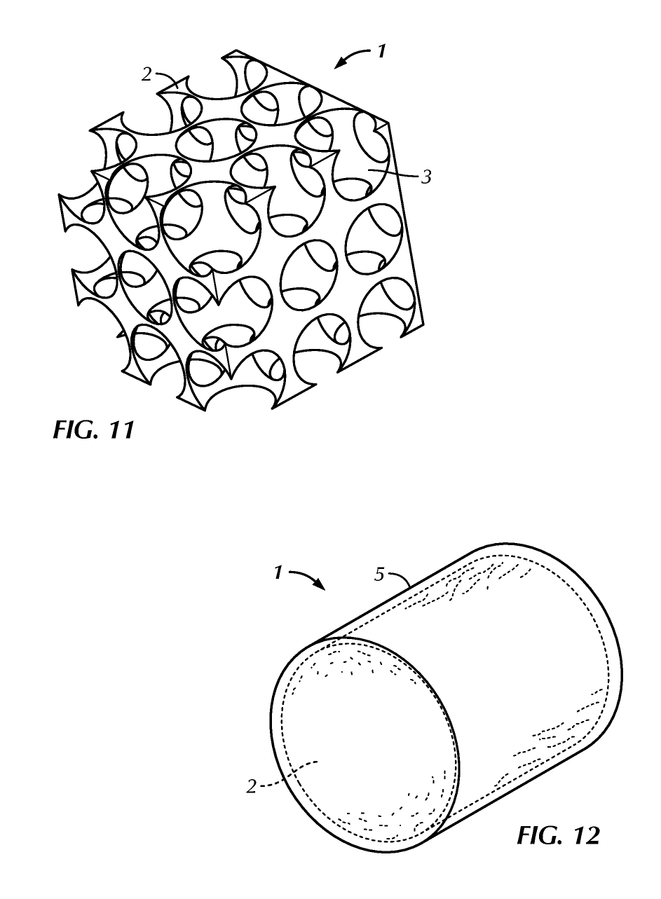

FIGS. 1 and 11 illustrate exemplary embodiments of the implant 1. The implant 1 may be any shape including, but not limited to, cylindrical (see FIG. 12), ring, crescent, screw, dog bone, barbell, circular, triangular and tubular. In one embodiment, the implant is cuboid shape as shown. Before implantation, in one embodiment, the implant 1 includes a compressed body 2 (e.g., a polymeric body) with open pores or cavities 3 and an envelope 5 in which said compressed body 2 is embedded. In one embodiment, said body 2 is embedded in or encased by the envelope 5 in an air-tight manner. In one embodiment, the body 2 is compressed by compressing the envelope 5.

In some embodiments, the body 2 is compressed from the uncompressed state (see FIG. 11) to the compressed state (see FIG. 12) by removing or evacuating at least some of the air from within the envelope 5. In one embodiment, at least some air within the body 2 is evacuated. In one embodiment, at least some air within the body 2 and the envelope 5 is evacuated. In one embodiment, substantially all of the air within the envelope 5 is evacuated in the compressed state. In one embodiment, the envelope 5 has a valve 6 for evacuation of air. In one embodiment, the valve 6 is an integrated nonreturn (e.g., one-way) valve. In one embodiment, the air is removed from the envelope 5 by applying a vacuum. For example, a vacuum may be fluidly attached to the area contained within the envelope 5 through the valve 6 to remove air from within the envelope 5 to reduce the volume of the body 2. In one embodiment, the air is removed from the envelope 5 to compress the body 2 by applying a force (e.g., squeezing) the envelope 5 in addition to or in place of a vacuum.

In one embodiment, the body 2 is comprised of a highly porous polymer foam. In one embodiment, the material of the body 2 is compressible rather than only deformation of the implant itself. In one embodiment, the body 2 is comprised of an elastomer, silicone and/or biodegradable material from the polylactide or polycaprolactide family. In one embodiment, the body 2 preferably has, in its uncompressed state, a degree of porosity of about 80% to about 95% wherein the degree of compression of the body 2 is about 5±2% for 80% porosity and is about 20±5% for 95% porosity. In one embodiment, the porosity of the body 2, in its uncompressed state, is greater than about 80% with pores larger than 1 mm.

The envelope 5 may be biodegradable, resorbable, water-soluble and/or otherwise degradable in body fluids. In one embodiment, the envelope 5 is comprised of a polyvinyl alcohol (PVA), starch or methylcellulose material. In one embodiment, the envelope 5 has a thickness between about 10 μm and about 500 μm. In one embodiment, the envelope 5 has a minimum thickness of about 100 μm. In one embodiment, the envelope 5 has a maximum thickness of about 300 μm.

In one embodiment, the envelope 5 is under negative pressure in the compressed state to keep the implant 1 compressed. The vacuum in the pores or cavities 3 of the body 2 may be about 10 mbar or less in the compressed state. Removing the vacuum, in some embodiments, allows the body 2 to expand toward its uncompressed state. In one embodiment, the body 2 returns completely to its uncompressed state after being implanted. In some embodiments, the body 2 does not fully return to a completely uncompressed state in use due to external limitations such as from the surrounding tissue. In one embodiment, the implant 1 does not fully occupy a space within the body when initially implanted in the compressed state to allow for easier and/or less invasive insertion into the body and subsequently fully occupies the space after a period of time (e.g., once the vacuum within the envelope is released) or at the least, increases in size.

FIG. 2 illustrates an embodiment of the implant 1 which differs from the embodiment of FIG. 1 in that the envelope 5 is surrounded by a protective sheath 7. The protective sheath 7 may be comprised of a thermoplastic material, preferably of polylactide (PLA) or polycaprolactone (PCL) in a dense form. In one embodiment, the protective sheath 7 is not permeable to water.

FIG. 3 illustrates the uncompressed body 4 before evacuating the pores or cavities 3 of an embodiment of the implant 1 of FIG. 1. In one embodiment, the pores or cavities 3 of said uncompressed body 4 have a size of about 10 μm to about 2 mm. In one embodiment, the pores or cavities 3 of the uncompressed body 4 have a size of about 1 mm. FIG. 11 illustrates another arrangement of the pores or cavities 3 in another embodiment of the implant 1. In some embodiments, the pores or cavities 3 are oriented in a regular array (e.g., aligned rows and columns as illustrated in FIG. 11). In one embodiment, the pores or cavities 3 are arranged in an irregular array. In one embodiment, the pores or cavities 3 are arranged randomly.

FIG. 4 illustrates an embodiment of the implant 1 which differs from the embodiment of FIG. 1 in that, for example, the envelope 5 has one region 8 which consists of a material with a dissolution rate D that is different (e.g., higher) than the dissolution rate d of the material of the remaining part 9 of the envelope 5.

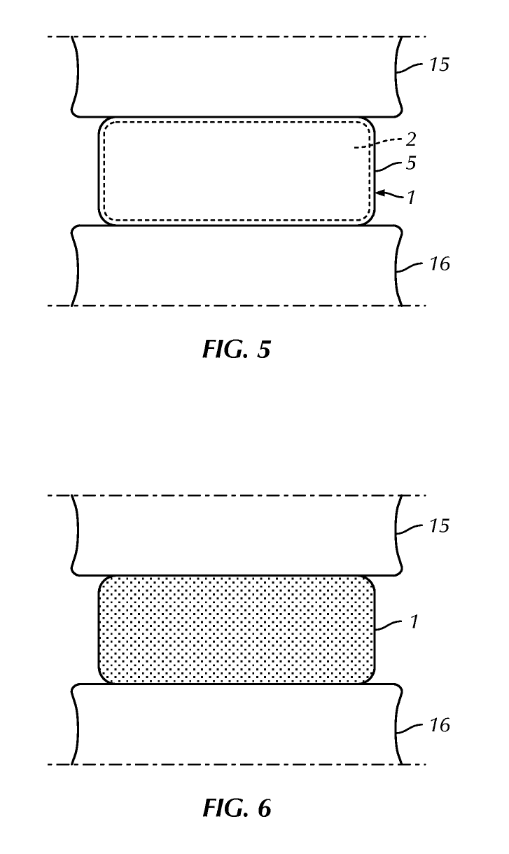

FIGS. 5 and 6 illustrate an application of the method for treating spine deformations using an implant 1 according embodiments shown in FIGS. 1 to 4 in the form of an interspinous spacer. In one embodiment, there is a the method comprising the following steps:

a) applying a spreading force to a first and second vertebral body 15, 16 which are adjacent to each other;

b) removing the intervertebral disk between said adjacent first and second vertebral body 15, 16;

c) inserting said implant 1 including said compressed body 2 and said envelope 5 into the intervertebral cavity; and

d) releasing said spreading force.

In one embodiment, after the envelope 5 has dissolved or degraded in vivo, air penetrates into the compressed body 2 which in turn expands to the shape of the uncompressed body 4 and/or as far as the physical boundaries of the intervertebral space permit and the implant 1 takes up its final implanted state (FIG. 6). In one embodiment, the re-expanding process causes the first and second vertebral body 15, 16 to move relative to each other in a direction parallel to the axis of the spinal column into a desired position relative to each other.

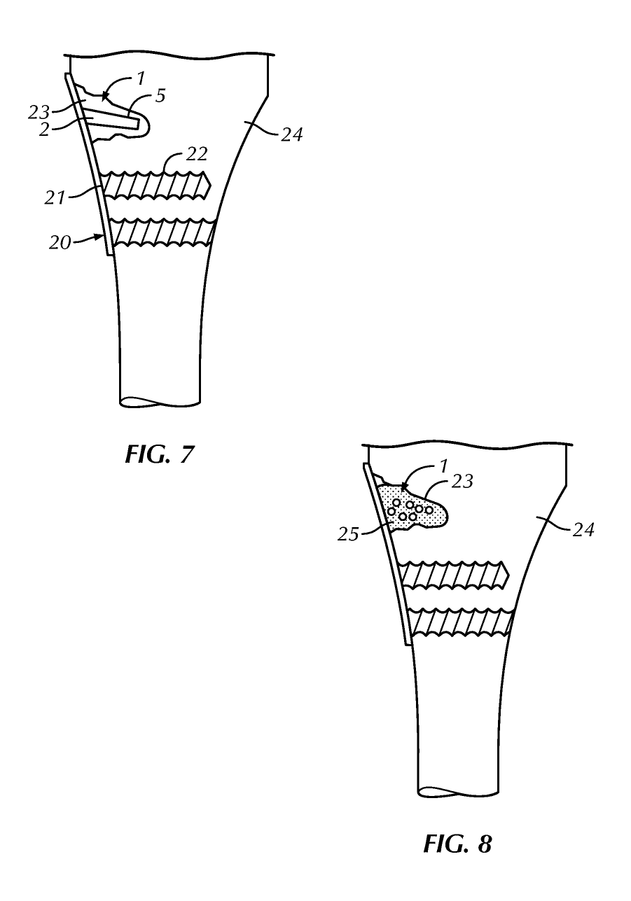

FIGS. 7 and 8 illustrate a bone fixation device 20 comprising a bone plate 21 to which an implant 1 is attached as a first bone anchoring means. In one embodiment, the bone plate 21 includes additional bone screws 22, for example, as second bone anchoring means. The implant 1 may be inserted into a cavity 23 which is e.g. drilled into the bone 24 or caused by a defect of the bone 24 in its unexpanded state. After fixation of the bone plate 21 to the bone 24 (e.g., by means of the fasteners 22) said envelope 5 of said implant 1 can dissolve or degrade and the compressed body 2 can expand to its final shape. In one embodiment, the implant 1 takes up its implanted state so as to form a further bone fastener 25 which is firmly fixed in said cavity 23 in said bone 24 (see FIG. 8).

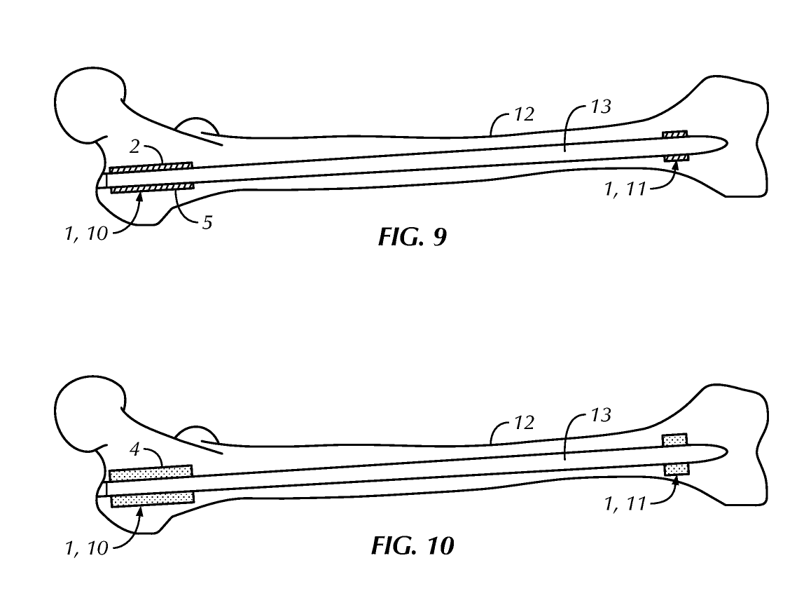

FIGS. 9 and 10 illustrate a further application of the implant 1 for internal locking of an intramedullary nail 13. In one embodiment, the implant 1 is used for distal and/or proximal locking of said intramedullary nail 13. The implant 1 may have a tubular shape so that a proximal and a distal locking collar 10, 11 may be positioned on the intramedullary nail 13. As illustrated in FIG. 9, in one embodiment, the intramedullary nail 13 is inserted into the medullary cavity of a bone 12, such as the femur, with a distal and a proximal implant 1 in its compressed state before implantation. In one embodiment, the implant includes an envelope 5 with the compressed body 2 embedded therein. After the implantation of one embodiment of the implant 1, the envelope 5 dissolves or degrades in vivo and air can penetrate into the compressed body 2. The compressed body 2 is expandable to the shape of the uncompressed body 4 and/or as far as the physical boundaries of the medullary cavity permit. Once the implant 1 has taken up its final implanted state as illustrated in FIG. 10, in one embodiment, the intramedullary nail 13 is firmly held in the medullary cavity by means of the proximal and distal locking collar 10, 11 formed by an implant 1 each.

FIG. 12 illustrates an implant 1 shown with the body 2 in the compressed configuration and sealed within the envelope 5.

Although the invention and its advantages have been described in detail, it should be understood that various changes, substitutions, and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, composition of matter, means, methods, or steps, presently existing or later to be developed that perform substantially the same function or achieve substantially the same result as the corresponding embodiments described herein may be utilized according to the present invention.

It will be appreciated by those skilled in the art that various modifications and alterations of the invention can be made without departing from the broad scope of the appended claims. Some of these have been discussed above and others will be apparent to those skilled in the art.

I/We claim:

1. A surgical implant comprising:
a body having a compressed state and an uncompressed state; and
an envelope containing the body in at least the compressed state, the envelope forming an air-tight seal around the body in the compressed state and the envelope being water-soluble and/or degradable in body fluids.

2. The implant according to claim 1, wherein the body includes a plurality of cavities.

3. The implant according to claim 2, wherein the cavities in an uncompressed state have a size of 10 μm to 2 mm.

4. The implant according to claim 2, wherein a vacuum in the cavities in the compressed state is 10 mbar or less.

5. The implant according to claim 4, wherein the envelope fully contains the body in the uncompressed state.

6. The implant according to claim 5, wherein the envelope includes a one-way valve for evacuation of air from the body from the uncompressed state to the compressed state.

7. The implant according to claim 1, wherein only part of a total area of the envelope is water-soluble or degradable in body fluids.

8. The implant according to claim 7, wherein a remaining part of the total area of the envelope comprises a high strength polymer.

9. The implant according to claim 1, further comprising a protective sheath at least partially surrounding the envelope and comprised of a thermoplastic material.

10. The implant according to claim 9, wherein the thermoplastic material is polylactide (PLA) or polycaprolactone (PCL).

11. The implant according to claim 1, wherein the body is comprised of a polymeric material.

12. The implant according to claim 1, wherein the body is comprised of a foam material.

13. The implant according to claim 1, wherein the envelope is comprised of polyvinyl alcohol (PVA) or methylcellulose.

14. The implant according to claim 1, wherein the envelope includes one or more regions comprised of a material having a dissolution rate D and a remainder of the envelope is comprised of material having a dissolution rate d<D.

15. The implant according to claim 1, wherein the envelope is surrounded by a protective sheath made of a material not permeable to water.

16. The implant according to claim 1, wherein the envelope has a minimum thickness of 10 μm.

17. The implant according to claim 1, wherein the envelope has a maximum thickness of 500 μm.

18. The implant according to claim 1, wherein the body in the uncompressed state has a degree of porosity larger than 80%.

19. The implant according to claim 1, wherein the body has a porosity and a degree of compression of 5±2% when the porosity is 80% and a degree of compression of 20±5% when the porosity is 95%.
